(19) United States
(12) Patent Application Publication
(10) Pub. No.: US 2026/0325354 A1
(43) Pub. Date: Oct. 22, 2026

(54) Automated Selective Laser Trabeculoplasty

(71) Applicant: Belkin Vision Ltd., Yavne (IL)

(72) Inventor: Zachary Sacks, Modiin (IL)

(21) Appl. No.: 19/111,347

(22) PCT Filed: Oct. 9, 2023
(86) PCT No.: PCT/IB2023/060104

§ 371 Date: Mar. 13, 2025

Related U.S. Application Data
(60) Provisional application No. 63/414,919, filed on Oct. 11, 2022.

(51) Int. Cl. A61F 9/009; A61B 3/00; A61B 3/117; A61B 3/14; A61F 9/008; G06T 7/00; G06T 7/11

(52) U.S. Cl. CPC A61F 9/009; A61B 3/0025; A61B 3/117; A61B 3/14; G06T 7/0012; G06T 7/11; A61F 2009/00844; A61F 2009/00868; G06T 2207/10004; G06T 2207/30041

(57) ABSTRACT

An apparatus (100) for medical treatment includes a gonioscope (118) having a distal face (120) for placement in proximity to an eye (142) of a patient, a proximal face (121) opposite the distal face, and multiple facets (119) between the distal and proximal faces. The apparatus also includes a camera (112) to capture an image of an anterior chamber (129), a laser (108) to generate a beam, a scanner (110) to direct the beam through the proximal face of the gonioscope, optics focusing the beam to impinge on the tissue with a cone angle no greater than 2O. A controller (132) processes the image of the anterior chamber so as to identify a locus (308) of a trabecular meshwork (150) in the eye and to control the scanner so as to direct the beam to impinge on the locus at multiple locations.

### CROSS-REFERENCE TO RELATED APPLICATION

This application claims the benefit of U.S. Provisional Patent Application 63/414,919, filed Oct. 11, 2022, which is incorporated herein by reference.

### FIELD OF THE INVENTION

The present invention relates generally to apparatuses and methods for treatment of the eye, and particularly to laser trabeculoplasty.

### BACKGROUND

Glaucoma is a group of eye diseases that lead to damage of the optic nerve. This damage is often caused by increased intraocular pressure (IOP) of the aqueous humor within the anterior chamber of the eye. This increased IOP may cause vision loss if left untreated.

One of the treatments used to reduce IOP is selective laser trabeculoplasty (SLT), which is described, for example, by Gazzard et al. in “Selective laser trabeculoplasty versus drops for newly diagnosed ocular hypertension and glaucoma: the LIGHT RCT” (NHS, volume 23, issue 31, June 2019, ISSN 1366-5278). In SLT, several laser shots are fired through the anterior chamber to the trabecular meshwork of the eye using a gonioscope. The laser irradiation of the trabecular meshwork improves the drainage of aqueous humor through the meshwork, thus alleviating the build-up of IOP within the eye.

The term “optical radiation” is used in the present description and in the claims to refer to electromagnetic radiation in any of the visible, infrared, and ultraviolet ranges of the spectrum.

### SUMMARY

Embodiments of the present invention that are described hereinbelow provide improved apparatuses and procedures for selective laser trabeculoplasty.

There is therefore provided, in accordance with an embodiment of the invention, an apparatus for medical treatment. The apparatus includes a gonioscope having a distal face, which is configured for placement in proximity to an eye of a patient, a proximal face opposite the distal face, and multiple facets extending between the distal and proximal faces. A camera is configured to capture, through the proximal face of the gonioscope, an image of an anterior chamber of the eye, and a laser configured to generate a beam of optical radiation. The apparatus further includes a scanner, which is configured to direct the beam through the proximal face of the gonioscope so that the beam reflects from a facet of the gonioscope into the anterior chamber to impinge on tissue in the anterior chamber, and optics configured to focus the beam to impinge on the tissue in the anterior chamber with a cone angle no greater than 2°. A controller is configured to process the image of the anterior chamber so as to identify a locus of a trabecular meshwork in the eye and to control the scanner so as to direct the beam to impinge on the identified locus at multiple locations around a circumference of the anterior chamber.

In a disclosed embodiment, the optics are configured to focus the beam so that the cone angle is less than 1.5°.

In a further embodiment, the camera has a depth of field sufficient to image all of the circumference of the anterior chamber through the gonioscope at a fixed focal setting. Typically, the optics are configured to direct the beam to impinge on all the multiple locations around the circumference of the anterior chamber at the fixed focal setting. Alternatively of additionally, the depth of field of the camera is at least 4 mm. Further alternatively, the depth of field is at least 3 mm, 2 mm, or 1 mm.

In yet another embodiment, in the image captured by the camera, the circumference of the anterior chamber is divided into multiple segments due to reflection of parts of the image from the multiple facets of the gonioscope, and the processor is configured to stitch together the multiple segments to generate an output image in which the locus of the trabecular meshwork appears as a continuous band.

In a disclosed embodiment, the distal face of the gonioscope includes a concave surface configured to contact a cornea of the eye. The apparatus may include a suction ring surrounding the gonioscope and configured to maintain a stable contact between the eye and the gonioscope.

There is also provided, in accordance with an embodiment of the invention, a method for medical treatment. The method includes positioning a distal face of a gonioscope in proximity to an eye of a patient, capturing through the gonioscope an image of an anterior chamber of the eye, processing the image of the anterior chamber so as to identify a locus of a trabecular meshwork in the eye and directing a beam of optical radiation emitted by a laser through the proximal face of the gonioscope so that the beam reflects from a facet of the gonioscope into the anterior chamber and impinges on the identified locus in the anterior chamber at multiple locations around a circumference of the anterior chamber, while focusing the beam to impinge on the tissue in the anterior chamber with a cone angle no greater than 2°.

There is additionally provided, in accordance with an embodiment of the invention, a method for medical treatment, which includes positioning a distal face of a gonioscope in proximity to an eye of a patient and capturing, through the gonioscope, an image of an anterior chamber of the eye. The image of the anterior chamber is processed so as to identify a locus of a trabecular meshwork in the eye. A beam of optical radiation emitted by a laser is directed through the proximal face of the gonioscope so that the beam reflects from a facet of the gonioscope into the anterior chamber and impinges on the identified locus in the anterior chamber at multiple locations around a circumference of the anterior chamber using a fixed focal setting of the beam at all the multiple locations around the circumference of the anterior chamber.

In a disclosed embodiment, capturing the image of the anterior chamber comprises capturing the image with a depth of field sufficient to image all of the circumference of the anterior chamber through the gonioscope at the fixed focal setting.

The present invention will be more fully understood from the following detailed description of the embodiments thereof, taken together with the drawings in which:

### BRIEF DESCRIPTION OF THE DRAWINGS

FIG. 1 is a schematic side view of an SLT apparatus, in accordance with an embodiment of the invention;

FIG. 2 is a schematic representation of a frontal image of an anterior chamber of an eye captured by a camera through a gonioscope, in accordance with an embodiment of the invention;

FIGS. 3A, 3B and 3C are schematic representations of segments of an anterior chamber angle in three successive stages of processing of a gonioscopic image, in accordance with an embodiment of the invention;

FIGS. 4A and 4B are sectional and frontal partial views of the eye, respectively, showing a beam of a laser of the SLT apparatus and indicating a depth of field of the camera, in accordance with an embodiment of the invention; and

FIG. 5 is a flowchart that schematically illustrates a method for performing an SLT procedure, in accordance with an embodiment of the invention.

### DETAILED DESCRIPTION OF EMBODIMENTS

### Overview

In an SLT procedure, a patient is seated in front of the SLT apparatus, which is aligned to the patient's eye. The patient must remain with his/her head in a fixed position and orientation with respect to the SLT apparatus while the laser beam is focused on and shifted across the trabecular meshwork by the operating ophthalmologist. As in other laser surgical procedures, the laser beam is focused precisely onto each point in the trabecular meshwork that is to be treated as indicated by the sharp focus of an aiming beam through a surgical microscope. (The proper location of the treatment beam corresponds with the focus of the surgical microscope.) Because of irregularities in the eye and in positioning of the gonioscope, the focal position often varies from point to point. Furthermore, to ensure that the laser beam is properly aimed and focused, it is necessary to refocus the microscope that the ophthalmologist uses to view the gonioscopic images. As a large number of laser pulses are fired into the trabecular meshwork with concomitant alignment and focusing of the surgical microscope and the laser beam, the procedure may be prolonged, taxing the stamina of the ophthalmologist performing the procedure, as well as the ability of the patient to keep his/her head in a fixed position and orientation.

There is thus a need to automate and shorten the duration of the SLT procedure while ensuring that its efficacy is maintained. Embodiments of the present invention that are described herein address this problem by employing a laser emitting a beam that is only loosely focused, with a small cone angle, for example less than 2°. This innovation is based on the realization that the effectiveness of laser trabeculectomy is not dependent on creating a precise intensity on the trabecular meshwork. The use of a loosely focused beam in the present embodiments provides a large depth of field for the laser beam, thus relaxing the requirements for focusing the beam and speeding up the procedure. Under these conditions, the same focal setting can be generally used around the entire circumference of the anterior chamber. This arrangement also makes it possible to use a camera with a large depth of field for aligning the laser beam.

The disclosed embodiments thus provide an apparatus for medical treatment, comprising a gonioscope having a distal face, which is configured for placement in proximity to an eye of a patient, a proximal face opposite the distal face, and multiple facets extending between the distal and proximal faces. A camera captures, through the proximal face of the gonioscope, an image of the anterior chamber of the eye. A laser generates a beam of optical radiation, and a scanner directs the beam through the proximal face of the gonioscope so that the beam reflects from a facet of the gonioscope into the anterior chamber to impinge on tissue in the anterior chamber. Optics focus the beam to impinge on the tissue in the anterior chamber with a cone angle no greater than 2°, and possibly less than 1.520 . A controller processes the image of the anterior chamber so as to identify the locus of the trabecular meshwork in the eye and to control the scanner so as to direct the beam to impinge on the identified locus at multiple locations around a circumference of the anterior chamber.

### System Description

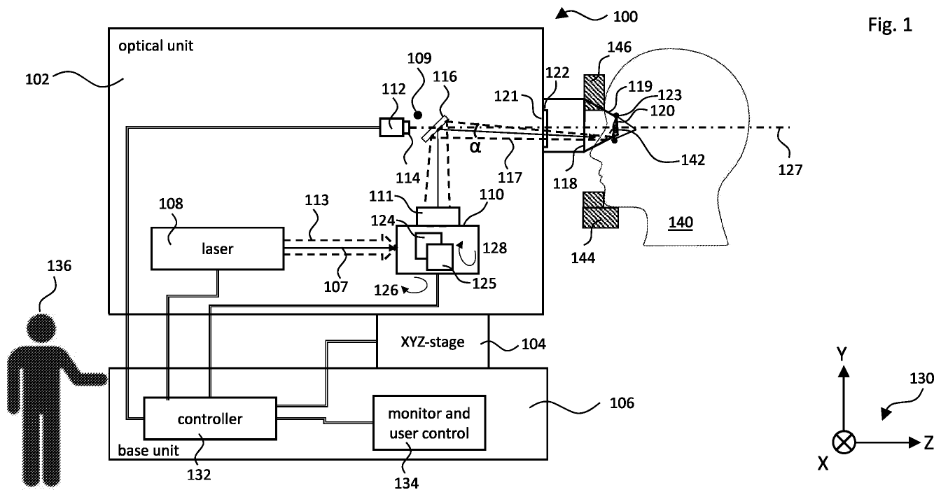

FIG. 1 is a schematic side view of a partially automated SLT apparatus 100, in accordance with an embodiment of the invention.

SLT apparatus 100 comprises an optical unit 102, an XYZ-stage 104, and a base unit 106. Optical unit 102 comprises a treatment laser 108 emitting a treatment beam 113 of optical radiation and an optional low-intensity integrated collinear aiming beam 107, which may include its separate focusing optics (not shown). The optical unit also comprises a scanner 110, a camera 112, a camera lens 114, a fixation point 109, and a beam combiner 116, which combines the optical paths of laser 108 and camera 112. Focusing optics 111 focus treatment beam 113 emitted by laser 108 and scanned by scanner 110 into a focused treatment beam 117 with a cone angle α no greater than 2°. In an alternative embodiment, cone angle α may be limited by optics 111 to no greater than 1.5°. For focusing beam 113, optics 111 may, in an alternative embodiment, be positioned between laser 108 and scanner 110. Optics 111 may also comprise a focusing mechanism.

In the present example, laser 108 comprises a frequency-doubled Nd:YAG Q-switched laser, emitting pulses at a wavelength of 532 nm with a pulse duration in the range of 1-10 nanoseconds, pulse frequency 1-100 Hz, and pulse energy ranging from 0.2 mJ to 2.6 mJ. Alternatively, any other suitable type of laser may be used, operating in either pulsed or CW mode.

Optical unit 102 further comprises a gonioscope 118, comprising multiple reflecting facets 119 arranged in a truncated cone between a distal face 120 and a proximal face 121, and an illumination ring 122. In the examples shown in the figures that follow, the gonioscope has four or six facets; but alternatively, the gonioscope may comprise any suitable number of facets or may have a continuous curved shape. Distal face 120 is concave and in certain embodiments is surrounded by a suction ring 123 to maintain a stable contact between the patient's eye and the gonioscope. Gonioscope 118 is collinear with and centered on an optical axis 127 of camera 112.

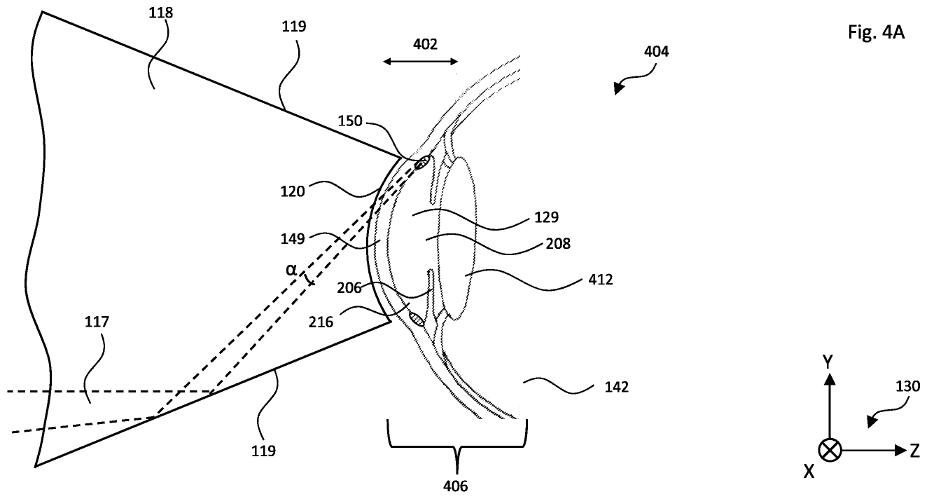

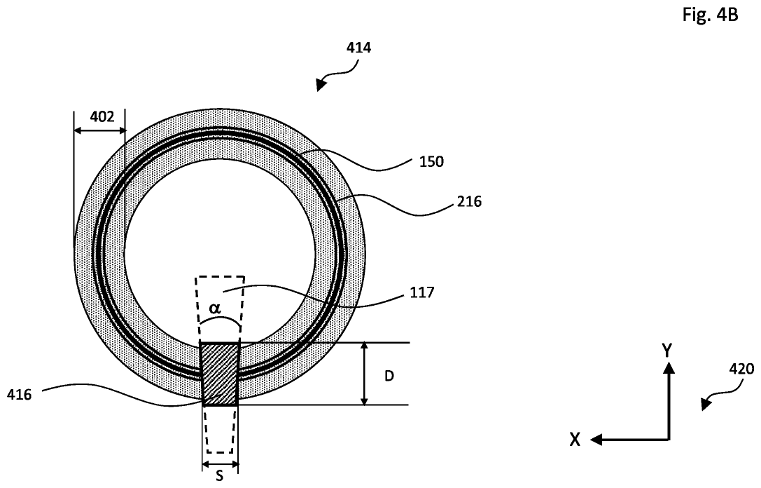

Scanner 110 comprises two galvanometer mirrors 124 and 125 rotating around two orthogonal axes (not shown for the sake of simplicity), with the rotations indicated by respective circular arrows 126 and 128. Scanner 110 is configured to direct beam 117 through proximal face 121 of gonioscope 118 so that the beam reflects from a facet 119 of the gonioscope through distal face 120 into an anterior chamber 129 (FIG. 4A) of an eye 142 in contact with the distal face so as to impinge on tissue in the anterior chamber.

XYZ-stage 104 moves optical unit 102 in the three linear orthogonal X-, Y-, and Z-directions, as indicated by Cartesian coordinates 130.

Base unit 106 comprises a controller 132, as well as a monitor and user control unit 134. Controller 132 is coupled to camera 112, laser 108, scanner 110, XYZ-stage 104, and monitor and user control unit 134. Alternatively, the monitor and/or the entire user control unit 134 and/or the controller may be integrated in the optical unit 102.

Controller 132 typically comprises a programmable processor, which is programmed in software and/or firmware to carry out the functions that are described herein. Alternatively or additionally, controller 132 comprises hard-wired and/or programmable hardware logic circuits, which carry out at least some of the functions of the controller. Although controller 132 is shown in the figure, for the sake of simplicity, as a single, monolithic functional block, in practice the controller may comprise a single chip or a set of two or more chips, with suitable interfaces for receiving and outputting the signals that are illustrated in the figure and are described in the text. Monitor and user control unit 134 comprises one or more visual displays and suitable input devices, such as a keyboard, joystick, and/or mouse, enabling an operator 136 to interact with SLT apparatus 100. (Details of monitor and user control unit 134 have been omitted from the figure for the sake of simplicity.)

For an SLT procedure, a patient positions his/her head 140 in front of optical unit 102, so that his/her eye 142 is in proximity to distal face 120 of gonioscope 118. Apparatus 100 typically comprises a chin rest 144 and a forehead rest 146 for enhanced stability of patient's head 140 during the procedure. Chin rest 144 and forehead rest 146 may be integrated into base unit 106 or attached to a table of apparatus 100. Fixation point 109 is provided for the patient to align and stabilize his/her eye 142.

Operator 136 observes eye 142 in an image captured by camera 112 and displayed on a monitor of unit 134. For this purpose, eye 142 may be illuminated by illumination ring 122, for example, although alternatively, other sorts of light sources may be used. Camera 112, together with lens 114, has a depth of field sufficient to image the entire circumference of anterior chamber 129 of eye 142 at a single focal setting of the camera. While observing the eye, operator 136 moves, with an input device such as a joystick, optical unit 102 in the X- and Y-directions so that optical axis 127 of camera 112 is aligned with eye 142. Operator 136 then moves optical unit 102 in the Z-direction to bring the concave surface of distal face 120 into contact with the cornea of eye 142 (FIG. 4A). Prior to making contact, a gel or other suitable contact material may be applied to cornea 149. In certain embodiments suction ring 123 maintains a stable contact between eye 142 and gonioscope 118.

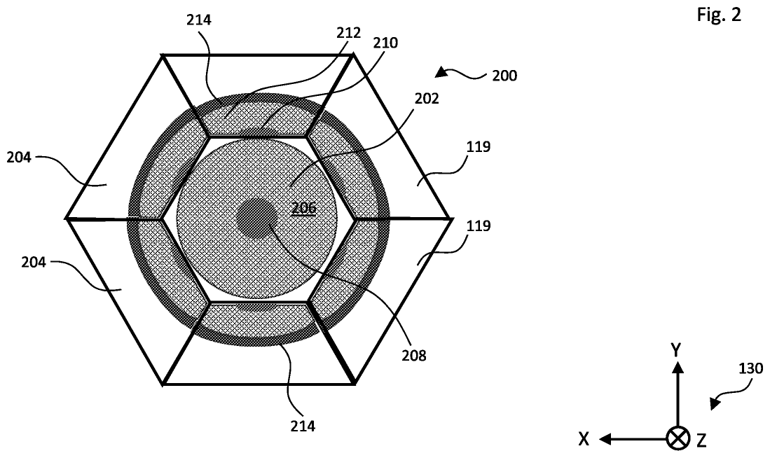

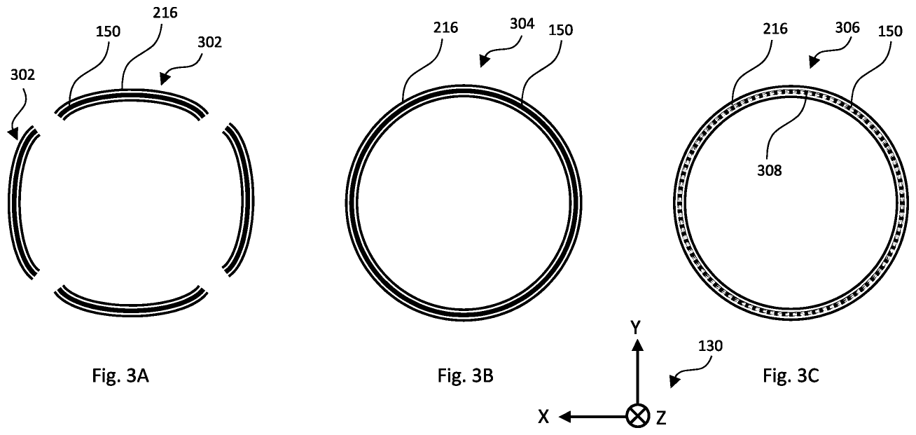

After the alignment process described hereinabove, operator 136 fine-tunes the XY-position of optical unit 102 so as to center eye 142 in the field of view of camera 112 and image an entire 360° field of view (for example as shown in FIG. 2). The same fixed focal setting is used around the entire circumference of anterior chamber 129, and there is no need to refocus camera 112 at different points around the circumference, even if the image is not perfectly sharp at all points. As will be further detailed with reference to FIGS. 3A-3C hereinbelow, controller 132 identifies the locus of the trabecular meshwork of eye 142 in an image captured by camera 112, and then directs scanner 110 to direct beam 117 to impinge on the trabecular meshwork at multiple locations around the circumference of the anterior chamber during the procedure. Before the actual firing of laser 108, operator 136 verifies the position of locus 308 on the trabecular meshwork and the alignment of the laser using aiming beam 107 displayed on the monitor. Once laser 108 is activated to emit beam 113, a typical procedure may take less than a minute and possible only a few seconds using a pulse frequency of 50-100 Hz and pulse energy of ~1 mJ. Prior to emitting treatment beam 113, aiming beam 107 may be swept over the target points with operation verified by operator 136 before proceeding to treatment mode.

FIG. 2 is a schematic representation of a frontal image 200 of the anterior chamber of eye 142 captured by camera 112 through gonioscope 118, in accordance with an embodiment of the invention.

Image 200 comprises both a direct image 202 of the anterior chamber and reflected images 204 reflected by facets 119 of gonioscope 118. In the embodiment shown in FIG. 2, gonioscope 118 comprises six reflecting facets 119; in alternative embodiments the number of facets 119 may be less or more than six, such as four, eight, twelve, or any other number of facets.

Direct image 202 comprises an image of an iris 206 and a pupil 208 of eye 142, without reflections from facets 119. Each reflected image 204 may comprise a partial iris image 212 and may comprise a partial pupil image 210. Furthermore, each reflected image 204 comprises an image segment 214 of the angle of the anterior chamber (as shown in FIG. 4A), which corresponds to the locus of a respective part of the trabecular meshwork. Due to the large depth of field of camera 112, image segments 214, reflected by respective facets 119, may be brought simultaneously into sufficient focus on the camera in all reflected images 204 to aim the weakly focused treatment beam 117, avoiding the need to re-focus the camera onto different parts of the anterior chamber angle and thus speeding up the procedure. Due to the optical construction of gonioscope 118, however, image segments 214 are distorted and separated from each other, as will be further detailed with reference to FIG. 3A, hereinbelow.

FIGS. 3A-3C schematically show image segments 302 of an angle 216 of the anterior chamber over a 360° circumference in three respective stages of processing of an image captured by camera 112, in accordance with an embodiment of the invention.

FIG. 3A shows image segments 302 of anterior chamber angle 216 captured through a gonioscope (similar to gonioscope 118 but with four reflecting facets). A trabecular meshwork 150 of the eye is located within angle 216. As indicated in FIG. 2 hereinabove, image segments 302, as captured by camera 112, are distorted from their actual shape, as well as separated from each other.

FIG. 3B is a schematic image 304 of an (in general) elliptical and continuous image of anterior chamber angle 216 and trabecular meshwork 150, wherein controller 132 has un-distorted and stitched image segments 302 in order to identify a locus of the trabecular meshwork.

FIG. 3C is a schematic image 306, in which controller 132 has identified a locus 308 (dotted line) of trabecular meshwork 150, along a circumference of the anterior chamber. Controller 132 will control laser 108 and scanner 110 to fire and direct laser beam 117 to impinge on the trabecular meshwork at multiple locations around locus 308.

FIGS. 4A and 4B schematically show a sectional partial view 404 and a frontal partial view 414 of eye 142, showing beam 117 of laser 108 and indicating a depth of field 402 of camera 112, in accordance with an embodiment of the invention.

Sectional view 404 in FIG. 4A comprises an anterior part 406 of eye 142. Anterior part 406 comprises iris 206, pupil 208, a cornea 149, an anterior chamber 129 (filled with aqueous humor), a lens 412, and trabecular meshwork 150 of eye 142. (Additional structures in anterior part 406 in the figure are not relevant to the current description, and have been left unlabeled for the sake of simplicity.) Angle 216 of anterior chamber angle 149 is located between cornea 149 and iris 206 and contains trabecular meshwork 150. Sectional view 404 further comprises a partial sectional view of gonioscope 118, showing parts of two facets 119 and distal face 120.

Beam 117 of laser 108 reflects from one of facets 119 of gonioscope 118 and impinges on trabecular meshwork 150 through cornea 149 and anterior chamber 129 (with refraction ignored for the sake of clarity). Due to the low value of cone angle α (less than 3°), beam 117 has a sufficient spot size to deliver laser energy to trabecular meshwork 150 over a sufficient depth for the entire meshwork around the 360° circumference, without the need to re-focus laser 108 during the procedure.

Camera 112 focuses on anterior chamber angle 216 and trabecular meshwork 150 with a sufficient depth of field 402 to capture the entire image 200 (FIG. 2) at a single focal setting of the camera. For example, the depth of field may be more than 1 mm, or more than 2 mm, or more than 3 mm, or more than 4 mm, or even more than 5 mm. Depth of field 402 is tied to the size of the blur circle of camera 112, meaning that the depth of field in this case refers to the ability of controller 132 to aim focused treatment beam 117 in the direction of trabecular meshwork 150 even when the trabecular meshwork is not in sharp focus as viewed by operator 136.

Frontal view 414 in FIG. 4B shows trabecular meshwork 150 and anterior chamber angle 216, shown in the XY-plane of Cartesian coordinates 130, indicated in the figure by X- and Y-axes 420. For the sake of this two-dimensional representation, laser beam 117 and depth of field 402 of camera 112 are indicated with their Z-directions flattened and reshaped into a circular shape in the XY-plane. The small cone angle α of beam 117 defines a treatment region 416 extending through trabecular meshwork 150, with a typical laser spot size S of 0.4 mm. As an example, a cone angle α of 1.5°yields a depth D of 3 mm for region 416 with a +10% change in the spot size over the region. Depth of field 402 for camera 112 may be 2 mm, 3 mm, 4 mm, or more. Having a camera depth of field 402 equal to or larger than the laser depth of focus D assures that, for a focused image on camera 112, beam 117 is also in focus.

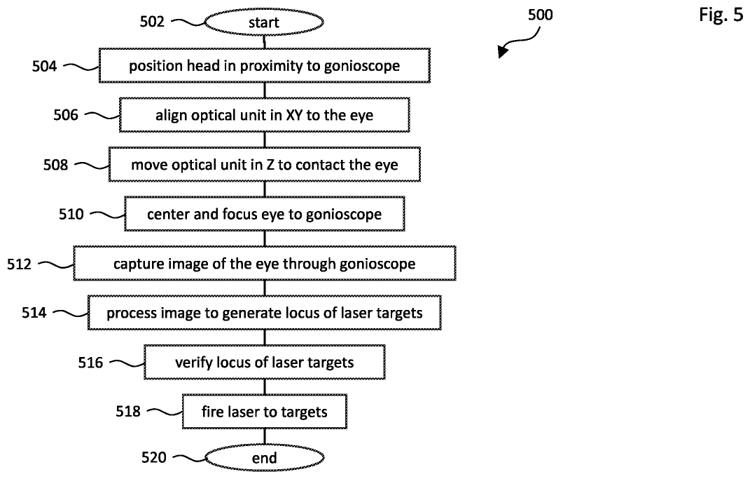

FIG. 5 is a flowchart 500 that schematically illustrates a method for performing an SLT procedure using SLT apparatus 100, in accordance with an embodiment of the invention.

The procedure starts in a start step 502. In a head positioning step 504, a patient positions his/her head 140 in proximity of gonioscope 118 (FIG. 1). In an alignment step 506, operator 136 aligns optical unit 102 with eye 142 in the XY-plane. In a Z-movement step 508, operator 136 moves optical unit 102 in the Z-direction so as to contact cornea 149 of eye 142 with distal face 120 of gonioscope 118. In a centering and focusing step 510, operator 136 centers and focuses optical unit 102 to position eye 142 at or near the center of the field of view of camera 112. In an image capture step 512, operator 136 captures with camera 112 an image of eye 142 through gonioscope 118.

In an image processing step 514, controller 132 processes the captured image to define locus 308 of trabecular meshwork 150 (FIG. 3C). In a target verification step 516, operator 136 views locus 308 on trabecular meshwork 150. Optionally, aiming beam 107 is fired around some or all the locus of target points and displayed on monitor 134 to verify correct laser operation. Provided that the locus coincides with the trabecular meshwork, the operator fires laser 108 to impinge on multiple points around the locus in a firing step 518. (If the locus identified by the controller does not coincide with trabecular meshwork 150, operator 136 may command SLT apparatus 100 to return to centering and focusing step 510 or adjust the locus manually.) The SLT procedure ends in an end step 520.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Apparatus for medical treatment, comprising:
a gonioscope having a distal face, which is configured for placement in proximity to an eye of a patient, a proximal face opposite the distal face, and multiple facets extending between the distal and proximal faces;
a camera configured to capture, through the proximal face of the gonioscope, an image of an anterior chamber of the eye;
a laser configured to generate a beam of optical radiation;
a scanner, which is configured to direct the beam through the proximal face of the gonioscope so that the beam reflects from a facet of the gonioscope into the anterior chamber to impinge on tissue in the anterior chamber;
optics configured to focus the beam to impinge on the tissue in the anterior chamber with a cone angle no greater than 2°; and
a controller, which is configured to process the image of the anterior chamber so as to identify a locus of a trabecular meshwork in the eye and to control the scanner so as to direct the beam to impinge on the identified locus at multiple locations around a circumference of the anterior chamber.

2. The apparatus according to claim 1, wherein the optics are configured to focus the beam so that the cone angle is less than 1.5°.

3. The apparatus according to claim 1, wherein the camera has a depth of field sufficient to image all of the circumference of the anterior chamber through the gonioscope at a fixed focal setting.

4. The apparatus according to claim 3, wherein the optics are configured to direct the beam to impinge on all the multiple locations around the circumference of the anterior chamber at the fixed focal setting.

5. The apparatus according to claim 3, wherein the depth of field of the camera is at least 1 mm.

6. The apparatus according to claim 5, wherein the depth of field of the camera is at least 2 mm.

7. The apparatus according to claim 6, wherein the depth of field of the camera is at least 3 mm.

8. The apparatus according to claim 7, wherein the depth of field of the camera is at least 4 mm.

9. The apparatus according to claim 3, wherein in the image captured by the camera, the circumference of the anterior chamber is divided into multiple segments due to reflection of parts of the image from the multiple facets of the gonioscope, and wherein the processor is configured to stitch together the multiple segments to generate an output image in which the locus of the trabecular meshwork appears as a continuous band.

10. The apparatus according to claim 1, wherein the distal face f the gonioscope comprises a concave surface configured to contact a cornea of the eye.

11. The apparatus according to claim 10, and comprising a suction ring surrounding the gonioscope and configured to maintain a stable contact between the eye and the gonioscope.

12. A method for medical treatment, the method comprising:
positioning a distal face of a gonioscope in proximity to an eye of a patient;
capturing, through the gonioscope, an image of an anterior chamber of the eye;
processing the image of the anterior chamber so as to identify a locus of a trabecular meshwork in the eye; and
directing a beam of optical radiation emitted by a laser through the proximal face of the gonioscope so that the beam reflects from a facet of the gonioscope into the anterior chamber and impinges on the identified locus in the anterior chamber at multiple locations around a circumference of the anterior chamber, while focusing the beam to impinge on the tissue in the anterior chamber with a cone angle no greater than 2°.

13. The method according to claim 12, wherein the cone angle of the beam impinging on the tissue is less than 1.5°.

14. The method according to claim 12, wherein capturing the image of the anterior chamber comprises capturing the image with a depth of field sufficient to image all of the circumference of the anterior chamber through the gonioscope at a fixed focal setting.

15. The method according to claim 14, wherein focusing the beam comprises using the fixed focal setting to direct the beam onto all the multiple locations around the circumference of the anterior chamber.

16. The method according to claim 15, wherein the depth of field is at least 1 mm.

17. The method according to claim 16, wherein the depth of field is at least 2 mm.

18. The method according to claim 17, wherein the depth of field is at least 3 mm.

19. The method according to claim 18, wherein the depth of field is at least 4 mm.

20. The method according to claim 14, wherein in the captured image the circumference of the anterior chamber is divided into multiple segments due to reflection of parts of the image from the multiple facets of the gonioscope, and wherein processing the image comprises stitching together the multiple segments to generate an output image in which the locus of the trabecular meshwork appears as a continuous band.

21. The method according to claim 12, wherein the distal face of the gonioscope comprises a concave surface configured to contact a cornea of the eye.

22. The method according to claim 21, and comprising providing a suction ring surrounding the gonioscope and maintaining a stable contact between the eye and the gonioscope.

23. A method for medical treatment, the method comprising:
positioning a distal face of a gonioscope in proximity to an eye of a patient;
capturing, through the gonioscope, an image of an anterior chamber of the eye;
processing the image of the anterior chamber so as to identify a locus of a trabecular meshwork in the eye; and
directing a beam of optical radiation emitted by a laser through the proximal face of the gonioscope so that the beam reflects from a facet of the gonioscope into the anterior chamber and impinges on the identified locus in the anterior chamber at multiple locations around a circumference of the anterior chamber using a fixed focal setting of the beam at all the multiple locations around the circumference of the anterior chamber.

24. The method according to claim 23, wherein capturing the image of the anterior chamber comprises capturing the image with a depth of field sufficient to image all of the circumference of the anterior chamber through the gonioscope at the fixed focal setting.
